(19) United States
(12) Patent Application Publication
(10) Pub. No.: US 2007/0014752 A1
(43) Pub. Date: Jan. 18, 2007

(54) Surface functionalization of polymeric materials

(76) Inventors: Krishnendu Roy, Austin, TX (US); Laxminariyan L. Raja, Austin, TX (US)

Correspondence Address: BAKER & BOTTS, 30 ROCKEFELLER PLAZA, 44TH FLOOR, NEW YORK, NY, 10112-4498, US

(21) Appl. No.: 11/483,019

(22) Filed: Jul. 7, 2006

Related U.S. Application Data
(60) Provisional application No. 60/697,480, filed on Jul. 8, 2005.

(51) Int. Cl. A61K 31/74

(52) U.S. Cl. 424/78.08

(57) ABSTRACT

The present invention relates to methods for functionalizing a surface, comprising exposing a surface of a polymeric material to an atmospheric pressure glow plasma discharge, wherein exposure to the plasma discharge functionalizes the surface of the polymeric material. The present invention further provides for methods for functionalizing a polymeric material, wherein the functionalized surface has conjugated thereto bioactive agents. The present invention is also directed to compositions comprising a functionalized surface with attached bioactive agents.

### CROSS REFERENCE TO RELATED APPLICATIONS

This application claims priority to U.S. Provisional Application No. 60/697,480 filed Jul. 8, 2005, the contents of which is incorporated in its entirety herein.

### STATEMENT REGARDING FEDERALLY FUNDED RESEARCH

The invention disclosed herein relates to work supported in part under grant number 6511817 in the division of Chemical and Transport Systems of the National Science Foundation. Accordingly, the U.S. government has certain rights in the invention.

### FIELD OF THE INVENTION

The present invention relates to methods for functionalizing a surface, comprising exposing a surface of a polymeric material to an atmospheric pressure glow (APG) plasma discharge, wherein exposure to the plasma discharge functionalizes the surface of the polymeric material. The present invention further provides for methods for functionalizing a polymeric material, wherein the functionalized surface has conjugated thereto bioactive agents. The present invention is also directed to compositions comprising a functionalized surface with attached bioactive agents.

### BACKGROUND OF THE INVENTION

### Biodegradable Delivery Particles

Biodegradable polymer particles, such as microparticles and nanoparticles such as biodegradable poly(lactide-co-glycolide) (“PLGA”) microparticles, are effective delivery vehicles for the controlled release of therapeutic compositions such as polypeptides, proteins, nucleic acids, vaccines, etc. Biodegradable polymer particles are also effective delivery vehicles for the controlled release of contrast and imaging agents in the human body. They also have applications in diagnostic and therapeutic imaging. However, before therapeutic compositions or contrast or imaging agents can be loaded onto a particle's surface, the surface must be functionalized. Examples of surface functionalization include the addition of negative charges or amine (NH₂) radical groups to a particle's surface.

There is increasing interest towards improving the potency of protein and DNA vaccines as alternatives for viral vectors because of safety issues (O'Hagan and Rappuoli, 2004, Drug Discov. Today 9, 846-854). Synthetic polymer based micro/nanoparticulate systems are being extensively researched in view of exciting results obtained in pre-clinical models (Langer et al., 1997, Adv. Drug Deliv. Rev., 28, 97-119). Recent evidence has shown that charged microparticle delivery systems that facilitate surface adsorption of proteins and DNA are a better alternative to traditional designs with encapsulation of proteins and DNA molecules (Singh et al., 2006, Curr. Drug Deliv., 3, 115-120). Surface adsorption of proteins is advantageous in terms of being an efficient process with increased loading, structural stability post surface adsorption, and finally in view of the promising results obtained in vivo with pre-clinical models (Singh et al., 2006, supra). Surface adsorption of proteins facilitates the option of encapsulation of soluble adjuvants inside the formulations to generate combinatorial delivery systems. This approach is the most recent approach adopted by multiple research groups (Chong et al., 2005, J. Control Rel., 102, 85-99; Kazzaz et al., 2006, J. Control Rel., 110, 566-573).

Current methods of functionalizing biodegradable polymer particles include wet chemical conjugation and simple adsorption processes. These methods suffer from poor reproducibility, inefficiency, and complex processing requirements. Also, the chemicals currently used for surface modification, e.g. cationic surfactants, are often toxic.

### Materials Processing With Plasma Discharges

Materials processing using glow discharge plasma technology has been researched extensively, particularly in the semiconductor and microelectronics industry for semiconductor chip material (Integrated Circuits, (IC)) processing and manufacturing (Economou, 2000, Thin Solid Films 365, 348-367; Graves and Kushner, 2003, J. Vacuum Sci. Tech. A 21, S152-S156). These plasmas are characterized by highly nonequilibrium chemical and thermal properties, but are constrained by the critical requirement for vacuum operation, which requires maintaining operating pressures from about 1 mTorr to about 10 Torr. At higher pressures, glow discharges are inherently unstable and tend to constrict and form undesirable streamers or thermal arcs. Traditional plasmas have operated at low pressure created by vacuum, making continuous operation difficult and hence made the overall process expensive because of vacuum equipment (Economou, 2000, supra). Consequently, use of these glow plasmas is most common in high-value-added applications where the materials are processed in a batch mode at low throughput volumes. Highly energetic ion dynamics at low pressures have also traditionally restricted glow plasma processing to “hard” materials (e.g., silicon-based materials in microelectronics applications). It has been difficult to apply these traditional low-pressure plasma processing techniques to biomaterials, because biomaterials are typically unstable at the high temperatures required for the techniques.

In view of avoiding the costly equipment and also to allow processing of large area of materials with low ion energy dynamics (plasma quality and the net results on the substrate that is treated), Atmospheric-Pressure Glow (APG) discharges have been studied extensively (Kanazawa et al., 1988, J. Phys. D-Appl. Phys., 21, 838-840; Kanazawa et al., 1989, Nucl. Instr. & Meth. Physics Res., 37-8, 842-845; Shenton and Stevens, 2001, J. Phys. D-Appl. Phys., 34, 2761-2768; Shenton et al., 2002, J. Polymer Sci. A Polymer Chem., 40, 95-109).

Several configurations are available for generation and stabilization of a glow discharge at atmospheric pressure and one of the ideal techniques has been to introduce dielectric barriers in between the electrode plates. This process is known as the dielectric barrier-atmospheric pressure plasma glow discharge (DB-APG) (Massines et al., 1998, J. Appl. Phys., 83, 2950-2957; Massines et al., 2003, Surface Coat. Technol. 174, 8-14). As shown in FIGS. 1 and 2, two parallel electrodes are held in close proximity with a gap in between of a few millimeters, and driven by a high voltage of higher than 1 kV at audio frequencies of 10 kHz. The dielectric material used was poly(carbonate). APG discharges have been found to be most stable in noble gases such Helium and Argon, but molecular working gases such as nitrogen can also be used (Okazaki et al, 1993, J. Phys. D-Appl. Phys. 26, 889-892).

Modulation of the reactivity of the plasma environment to increase the reactivity is achieved by using other additive gases such as ammonia and oxygen (volume %) to extend the applications in materials processing (Gherardi and Massines, 2001, IEEE Trans. Plasma Sci., 29, 536-544). Varying parameters have been studied in terms of the dielectric barrier layer thickness, dielectric constant, voltage and frequency and the gap spacing towards maintaining a stable plasma glow discharge (Massines and Gouda, 1998, J. Phys. D-Appl. Phys., 31, 3411-3420). The similarities of the plasma characteristics in terms of glow discharge like chemistries in comparison to low pressure, vacuum mediated plasma discharge has been studied using optical emission spectroscopy and modeling studies (Shin and Raja, 2003, J. Appl. Phys., 94, 7408-7415).

There is a need in the art to safely and reproducibly associate therapeutic, diagnostic and/or imaging compounds to biodegradable compounds. The present invention addresses this need by harnessing materials processing technology, in a manner not previously contemplated or expected to be effective.

### SUMMARY OF THE INVENTION

Thus, the present invention is drawn to a method of functionalizing a polymeric surface, for example to provide a biodegradable polymeric drug delivery system. To that end, APG plasma glow discharge can be used to functionalize the surface of a polymeric material, to which a bioactive agent, such as a small molecule drug, a protein drug, a polypeptide drug, a peptide drug, a DNA drug, a RNA drug, an oligonucleotide drug, an immunomodulatory agent, a vaccine or a contrast or imaging agent, can be conjugated.

Thus, the present invention relates to the use of flow-through atmospheric pressure glow (“APG”) plasma discharges to functionalize the surface of a polymeric material. Advantages of APG discharges include the (1) highly non-equilibrium chemical and thermal property of the plasma (similar to classical low-pressure glow discharges); (2) high degree of uniformity over large areas and volumes (without constriction and the resulting streamer or arc formation); (3) relatively low ion energetics; and (4) one-atmosphere operation. Atmospheric pressure operation, among other things, eliminates the need for expensive vacuum equipment and allows for the processing of large-area material surfaces and volumes. Thermal non-equilibrium in APG plasmas can ensure, among other things, near-room temperature operation, permitting the processing of bioactive materials in the presence of highly temperature sensitive biological molecules like proteins, peptides, and DNA. It is readily apparent that such biological molecules include nucleic acids generally, as well as carbohydrates.

Thus, the present invention relates to methods for functionalizing a surface, comprising exposing a surface of a polymeric material to an atmospheric pressure glow plasma discharge, wherein exposure to the plasma discharge functionalizes the surface of the polymeric material.

The present invention further provides for methods of functionalizing a polymeric material comprising exposing a surface of a polymeric material to an atmospheric pressure glow plasma discharge, wherein exposure to the plasma discharge functionalizes the surface of the polymeric material. In a particular embodiment, the polymeric material is a biopolymer. In a specific embodiment, the biopolymer is wherein the biopolymer is poly(lactide-co-glycolide). In a preferred embodiment, the polymeric material is a biopolymer particle formed of poly(lactide-co-glycolide).

In another embodiment, the polymeric material is a two-or three-dimensional surface.

The present invention further provides for a method of functionalizing a polymeric surface, comprising entraining the polymeric material into a gas phase to form a suspended polymeric material; introducing the suspended polymeric material into a volume of the atmospheric pressure glow plasma discharge; and collecting the suspended polymeric material from the atmospheric pressure glow plasma discharge. The present method can further include loading the surface of the functionalized polymeric material with a bioactive agent.

In particular embodiments, the bioactive agent comprises a small molecule drug, a protein drug, a peptide drug, a DNA drug, a RNA drug, an oligonucleotide drug, an immunomodulatory agent, a vaccine antigen or a contrast or imaging agent. In an alternative embodiment, the bioactive agent is a vaccine antigen. In other embodiments, the bioactive agent further comprises a contrast or imaging agent.

The present invention also provides a composition comprising a polymeric material having a surface that has been exposed to an atmospheric pressure glow plasma discharge resulting in the presence of charges or charged radical groups on the surface of the polymeric material. In a further aspect, this composition additionally comprises a bioactive agent associated with the charges or charged radical groups on the surface of the polymeric material.

As provided for in a specific embodiment, the bioactive agent comprises at least one bioactive agent comprising a small molecule drug, a protein drug, a peptide drug, a DNA drug, a RNA drug, an oligonucleotide drug, an immunomodulatory agent, a vaccine antigen or a contrast or imaging agent. In a particular embodiment, the bioactive agent comprises a protein drug.

The present invention further provides for a composition comprising a polymeric material having an interior and having a surface that has been exposed to an atmospheric pressure glow plasma discharge resulting in the presence of charges or charged radical groups on the surface of the polymeric material; a first bio-active agent associated with the charges or charged radical groups on the surface of the polymeric material; and a second bioactive substance disposed within the interior of the polymeric material.

In a particular embodiment, the bioactive agent comprises at least one bioactive agent comprising a small molecule drug, a protein drug, a peptide drug, a DNA drug, a RNA drug, an oligonucleotide drug, an immunomodulatory agent, a vaccine antigen or a contrast or imaging agent. In a specific embodiment, the first bioactive agent comprises a vaccine antigen; and the second bio-active agent comprises an immunomodulatory agent that regulates immune response to the vaccine antigen.

In a particular embodiment of the composition of the present invention, the polymeric material is a biopolymeric particle of poly(lactide-co-glycolide); the first bioactive agent comprises a vaccine, and the second bioactive agent comprises an immunomodulatory agent that regulates immune response to the vaccine.

### BRIEF DESCRIPTION OF THE DRAWINGS

A more complete understanding of this disclosure may be acquired by referring to the following description taken in combination with the accompanying figures in which:

FIG. 1 is a schematic illustration of a set up for the plasma chamber to operate at atmospheric pressure and room temperature, according to a specific embodiment of the present invention.

FIG. 2 is a schematic diagram of a combinatorial system for therapeutic drug delivery. Soluble adjuvant (LTR ligands) encapsulated in microparticles are surface modified using solvent-free, atmospheric glow discharge (APG) plasma, to make anionic microparticles for subsequent therapeutic drug adsorption to make a single injectable formulation of adjuvants and therapeutic drugs.

FIG. 3 is a photograph of a dielectric-barrier atmospheric pressure glow (“DB-APG”) plasma discharge in helium gas according to a specific embodiment of the present disclosure. The plasma region is distinguished by a faint bluish glow (arrow).

FIG. 4 is a time-averaged spectrum from a pure helium APG, according to a specific embodiment of the present invention.

FIG. 5A is a false color intensified charge coupled device (ICCD) image of a uniform APG discharge, according to a specific example embodiment of this disclosure. The image was taken with an exposure (gate) time of 300 ns.

FIG. 5B is a false color ICCD image of a non-uniform APG discharge with streamer and filamentary arc formation, according to a specific example embodiment of this disclosure.

FIG. 6 is a scanning electron microscopy image of unmodified PLGA particles (average diameter approximately 1.5 microns), which show that the microparticles with smooth surfaces are a polydisperse size distribution. The size analysis was verified by a dynamic light scattering technique (not shown).

FIG. 7 is scanning electron microscopy image of plasma modified microparticles, showing a smooth surface comparable to the unmodified microparticles as shown in FIG. 6.

FIG. 8 is a graph of zeta potential measurements for APG plasma-processed PLGA microparticles and a control group of unprocessed particles, according to a specific example embodiment of this disclosure. Double emulsion PLGA microparticles (2.5 mg or 5 mg) were air dried on frosted microscopic glass slides and subsequently treated with helium plasma discharge. The zeta potentials are an average of 11 individual modifications batches for both 2.5 and 5 mgs of microparticle deposition with standard error bars. The unmodified PLGA microparticle controls are average of N=7. Two-sided T tests with unequal variances gave a P value of <0.001 for differences between the unmodified and the plasma modified surface charge analysis.

FIG. 9 is a graph showing increased zeta potentials with increased time exposure of APG-modified microparticles.

FIG. 10 is a graph showing that freeze drying of plasma-modified microparticles retained the anionic charges deposited with only slight variations with the absolute zeta potentials. Five and 2.5 milligrams of PLGA microparticles were modified with exposure to APG discharge. Control PLGA was directly used after freeze drying, so there are no two bars representing zeta potentials for the control batch.

FIG. 11 is scanning electron microscopy image of protein-loaded plasma-modified PLGA microparticles, which do not show macroscopic structural changes on the surface of the microparticles. The images are comparable in features to the unmodified and the plasma-modified microparticles in FIGS. 6 and 7.

### DETAILED DESCRIPTION

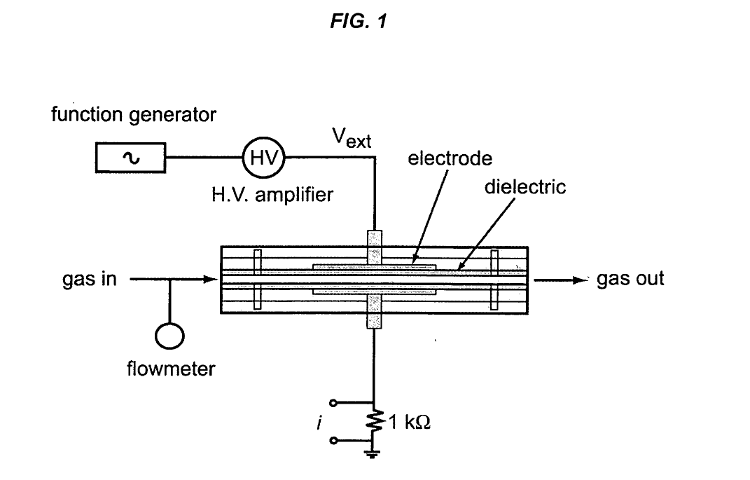

The present disclosure, according to one embodiment, relates to the use of flow-through atmospheric pressure glow (“APG”) plasma discharges to functionalize the surface of a particulate biopolymer (FIG. 1). Advantages of APG discharges include the (1) highly non-equilibrium chemical and thermal property of the plasma (similar to classical low-pressure glow discharges), (2) high degree of uniformity over large areas and volumes (without constriction and the resulting streamer or arc formation), (3) relatively low ion energetics and (4) one-atmosphere operation.

### Polymeric Materials For Functionalization

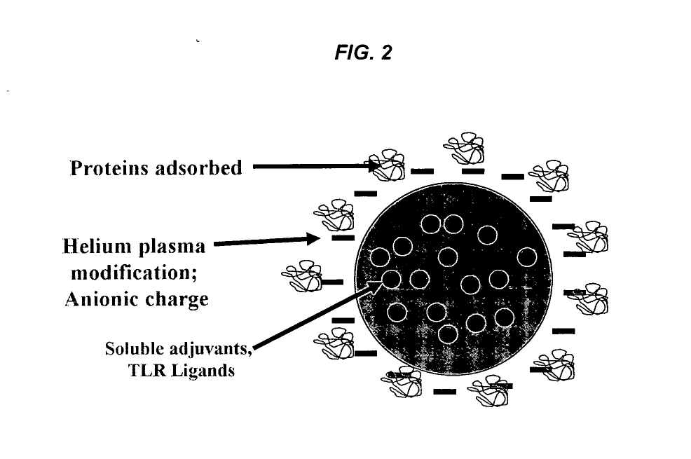

Surface functionalization of a polymeric material may be used, among other things, to prepare the polymeric surface to accept drugs, vaccines, and/or contrast agents. Functionalization may involve the addition of positive or negative charges or charged radical groups to the surface of the particle (FIG. 2). Drugs or contrast agents may be attached to the positive or negative charges or charged radical groups, and the polymeric material may then be used as delivery vehicles for the attached drugs or agents.

The material to be functionalized is typically a polymeric material. In particular embodiments, the polymeric material is a biopolymer. In some embodiments, the biopolymer is a biodegradable polymer. Biodegradable polymers include, without limitation, PMMA, poly(lactide-co-glycolide) (PLGA), polylactic acid (PLA), polyglycolic acid (PGA), polycaprolactone (PCL), or polyethylene glycol (PEG).

“Particles.” In a particular embodiment of the invention, the polymeric material is a biopolymer “particle,” which can be a microparticle or nanoparticle. A particle maybe formed from any biocompatible material, for example, silicon and silicon derivatives. According to a specific embodiment, polymeric material that is functionalized is a biodegradable poly(lactide-co-glycolide) (PLGA) microparticle. PLGA microparticles may be used as a slow-release drug delivery vehicle to achieve targeted drug delivery, reducing drug side effects, and the need for multiple drug administrations. Both protein and DNA-based vaccine antigen can be delivered by loading the protein or DNA onto the particles' surface. In addition, a plasma-surface functionalized particle of the present invention may be capable of targeting specific cells or tissues, such as diseased cells or tissues.

According to one embodiment, a microparticle with a porous shell structure and hollow core may be used. Small molecule drugs such as, for illustration and not limitation, immunomodulatory agents may be encapsulated in the hollow core of the microparticle. After appropriate surface modification of the particle, another protein or DNA drug, such as a vaccine antigen may be loaded onto the outer surface of the microparticle. In the human body, the drugs attached to the surface of the particle may be released immediately, while the drugs in the hollow core may diffuse out of the porous shell at a sustained rate.

“Scaffold.” In an alternative embodiment, the polymeric material to be functionalized may be a two- or three-dimensional surface, such as, without limitation, a scaffold. As used herein, “scaffold” refers to an artificial, biocompatible malleable structure that can be used to deliver therapeutic compositions, e.g., proteins, peptides, nucleic acids, viruses, etc., into the body, to support and direct the growth of new cells of an organ or tissue. In addition, scaffold can be used to support cells that are implanted or “seeded,” and which can support three-dimensional cell growth, such as tissue or organ growth or regeneration.

Scaffolds can be of natural or synthetic materials, and may be permanent, bioerodable or bioresorbable. Examples of natural scaffold materials include collagen, some linear aliphatic polyesters, chitosan, and glycosaminoglycans such as hyaluronic acid. Commonly used synthetic bioerodable scaffold materials include polylactic acid (PLA), polyglycolic acid (PGA); poly (lactide-co-glycolide) (PGLA) and polycaprolactone (PCL). Scaffolds generally have a high porosity to facilitate cell seeding and diffusion throughout the structure.

In a particular embodiment, the material can be a two- or three-dimensional surface such as, without limitation, a polymeric biomaterial for prostheses, or other polymer. In an alternate embodiment, the polymeric material used in non-biomedical applications may be non-polymer. By way of example and not of limitation, non-polymer materials that may be functionalized include, e.g., metals (stainless steel, cobalt-chromium alloys etc., ceramics, carbon materials etc.).

### Bioactive Agents

Bioactive agents are attached to the functionalized surface of the polymeric surface, e.g., to a microparticle, nanoparticle, scaffold, etc. Bioactive agents include, without limitation, a small molecule drug, a protein drug, a peptide drug, a DNA drug, a RNA drug, an oligonucleotide drug, an immunomodulatory agent, a vaccine antigen or a contrast or imaging agent.

The present invention also may be used for simultaneous noninvasive monitoring of a bioactive agent and the polymeric material, following administration. In a particular embodiment, imaging contrast agents are attached to the functionalized surface of the polymeric material separately or incorporated along with a bioactive agent. Examples of imaging contrast agents include quantum dots, gold nanoparticles or Gadalonium-diethylenetriaminepentaacetate (Gd-DTPA) (for use in magnetic resonance imaging (MRI)) and simultaneously image the, e.g., particles or scaffold in the body as they deliver and/or release the bioactive agent. Among other things, this allows the evaluation of the efficacy of the particle, for example, in reaching the target cells, intracellular uptake, and subsequent bioactive agent release. In certain embodiments, Gadalonium may be chemically conjugated to the device surface or loaded along with a bioactive agent.

### Functionalizing the Material

According to one embodiment, the material to be functionalized is suspended in a feed gas that is then introduced to the plasma discharge volume. Alternatively, the material is suspended in liquid, arranged in a thin liquid layer, or placed on a moving dielectric belt that passes through the plasma discharge volume. The surface of the material is functionalized as it passes through the APG plasma discharge. By way of explanation, and not of limitation, charged and radical species in the plasma interact with the material and functionalize its surface. Trace impurity species in the feed gas, such as trace amounts of oxygen, may also contribute to negative charge deposition on the material's surface. When the functionalized material emerges from the plasma discharge volume, it is collected. If the material is a particulate suspended in the gas phase, it may be collected by bubbling the particle-laden gas through 1 mM KCl. The functionalized materials are subsequently loaded with drugs, vaccines, or contrast agents. If necessary, particulates are then separated from the liquid through standard filtration and centrifugation techniques.

### Working Gasses

The APG plasma is sustained in a base working, inert gas that comprises helium, argon, nitrogen, or another diluent gas. By way of explanation and not of limitation, diluent gases are preferred as base working gases, because diluent gases often support the most stable and uniform plasma discharges. A discharge that is in streamer or thermal arcing mode may not desirable for the materials processing applications disclosed here. By way of explanation and not of limitation, when the working gas is a pure diluent working gas, pure negative charges are deposited on the surface of particles exposed to the plasma discharge.

According to certain embodiments of the present invention, the base working gas may be modified by adding a small amount (e.g., about a few percent by volume) of another gas. By way of explanation and not of limitation, the small amount of additive gas creates a reactive environment for processing the particles but does not affect the stability of the plasma. When plasma is sustained in a mixed gas, radical groups and electrical charges are deposited on the surface of particles exposed to the plasma. Examples of mixed gases that yield a positive charge deposition include nitrogen with ammonia (NH₃) additive and helium with ammonia additive. A positively charged functionalized surface is used to conjugate negatively charged agents such as, by way of example and not limitation, nucleic acids. By way of explanation and not of limitation, the APG plasma efficiently decomposes NH₃ to produce molecular and atomic fragments including NH₂, NH, N, H, and their corresponding ionic forms. These “daughter” radical species can subsequently be transported to the particle or surface to be functionalized, and functionalize the surface to form, for example, grafted amine groups. Other examples of additive gases are oxygen and fluorine.

### Measuring the Extent of Surface Functionalization

According to certain embodiments of the present disclosure, particles that have been exposed to an APG plasma discharge may be analyzed to determine the resulting deposition of surface charge and surface functional groups. A zeta potential measurement instrument may be used to measure the effective mobility of the particles in a known and well-characterized liquid medium. Zeta potential is a measurement of surface charge state. To determine the extent that a particle has been functionalized, the zeta potential of an unprocessed particle may be compared to the zeta potential of a processed particle. For example, NH₂ functionalization yields positive surface charge, and depending upon the extent or efficacy of amine modification, the zeta potential of an NH₂ functionalized particle may be increasingly positive compared to an unprocessed control particle.

One of skill in the art would understand that any method for surface analysis may be used to determine the deposition of the surface charge and surface functional groups. In a particular embodiment of the present invention, plasma-processed microparticles may be analyzed using elemental surface analysis techniques such as X-ray photoelectron spectroscopy (“XPS”), also known as Electron Spectroscopy for Chemical Analysis (ESCA), and imaging techniques such as scanning electron microscopy (“SEM”). XPS/ESCA provides a detailed understanding of the chemical state of the plasma-modified microparticle surface. SEM imaging provides insight into possible plasma-induced structural damage to the microparticles.

### Plasma Operating Parameters

The APG plasma can be generated in any of several configurations. According to one embodiment of the present disclosure, the APG configuration involves a dielectric-barrier discharge (“DB-APG”). A DB-APG may be generated by arranging two parallel electrodes in close proximity, usually a few millimeters apart. One or both of the electrodes are covered by a dielectric layer, such as, for example, polycarbonate. The electrodes are driven by a high voltage power supply at high audio. For example, voltage of approximately 1 kV and audio of approximately 10 kHz are appropriate.

The APG plasma is operated within a narrow range of parameters to create uniform and stable plasma discharges that are not unduly disrupted by APG plasma-particle interactions. By way of explanation and not of limitation, the presence of particles can significantly affect plasma stability boundaries and greatly increase the propensity for plasma constriction and streamer filamentary arc formation. Plasma stability boundaries may be affected by a number of factors, such as particle size, particle number densities, and other plasma operating parameters known to those skilled in the art.

According to certain embodiments, electrical characterization may be used to evaluate the plasma. Electrical characterization is based on discharge voltage-current measurements. The shape and the magnitude of current waveforms aid in the prediction of important plasma properties, such as the plasma mode (Townsend versus glow mode), plasma intensity, and the propensity for glow-to-arc transitions. Also, changes in the plasma state during particle processing are monitored through comparison of the discharge voltage-current waveforms of well-studied non-reactive APG plasma with APG plasmas in mixtures of gases and in the presence of suspended particles. Furthermore, waveform characteristics such as random current pulse formation, which are indicators of glow-to-arc transitions, may be useful to establish stability boundaries for the APG plasma in the particle processing environment. Electrical characterization may also yield insights useful for scale up in a manufacturing setting.

According to certain embodiments, time-resolved optical imaging may allow direct observation of the APG plasma structure during materials processing. An intensified charge coupled device (ICCD) camera system with about a 768×494 pixel resolution and an intensifier that can be gated down to about 100 ns at a framing rate of about 1 kHz may be satisfactory to provide time-resolved images of the discharge. ICCD imaging of an APG plasma under processing conditions may provide evidence of the impact of particle processing on the structure and uniformity of plasma.

According to certain embodiments, optical emission spectroscopy may provide evidence of the chemical structure of the APG plasma. Line emission from atomic species and band emission from molecular species may be identified and used to detect the species' absence or presence in the discharge. Line and band emission may also indicate the relative densities of the species under different discharge conditions. Through proper calibration and detailed measurements of the line and band shapes, additional information such as the absolute number densities of species, electron temperatures, and gas temperatures may also be estimated. The relative intensities of these emission bands under different processing conditions may be monitored to infer the relative densities of these species and correlated with the attributes of the particle functionalization. A time-resolved spectra from the plasma may be obtained using a 0.25 m spectrograph with a gated intensified linear diode array detector. In this way, light may be collected from the center of the discharge and transferred to the spectrograph using, for example, a 100 micron core optical fiber.

### Advantages And Uses of the Invention

APG plasma discharge may enable continuous materials processing without the need to break a vacuum to load drugs and agents onto the functionalized material. Room temperature operation does not melt delicate biopolymer materials during processing. Because the process does not involve toxic byproducts or liquid wastes, it is environmentally benign, and by avoiding the use of chemical entities to modify the surface (e.g., surfactants), the product may be a relatively safe vehicle for drug delivery. Another advantage of APG plasma processing is that the low ion impact energies in APG plasmas may reduce structural damage to the material's surface. The APG plasma processing technique can be high throughput, reproducible, efficient, and scalable for, among other things, pharmaceutical manufacturing needs. It may also be capable of being made into an on-line or automated process.

In certain embodiments, a microparticle may be used as a multi-agent, or combinatorial, drug delivery system. By way of explanation and not limitation, the role of one multi-agent may be to modulate the immune system's reaction to another multi-agent, such a protein or DNA vaccine also delivered by the molecule.

To facilitate a better understanding of the present disclosure, the following examples of specific embodiments are given. The following examples do not limit or define the entire scope of the invention.

### EXAMPLES

### Example 1

### Generation of An APG Plasma Discharge

Materials. Polymers And Reagents

PLGA Resomer® RG502H, RG503H was purchased from Boehringer Ingelheim, Germany (inherent viscosity (I.V.)=0.16-0.2 dl/g, MW, approximately 11,000 Da, obtained from the inherent viscosity vs. molecular weight correlation sheet)). Poly (vinyl alcohol) PVA, MW approximately 31,000 Da (approximately 88% hydrolyzed) was purchased from Fluka. Ovalbumin and Lysozyme proteins were purchased from Sigma-Aldrich (St. Louis, Mo.). Micro BCA kit for protein analysis was purchased from Pierce Biotechnology (Rockford, Ill.). All other lab supplies were procured from Fischer Scientific Inc (Pennsylvania, USA).

APG Plasma Discharge

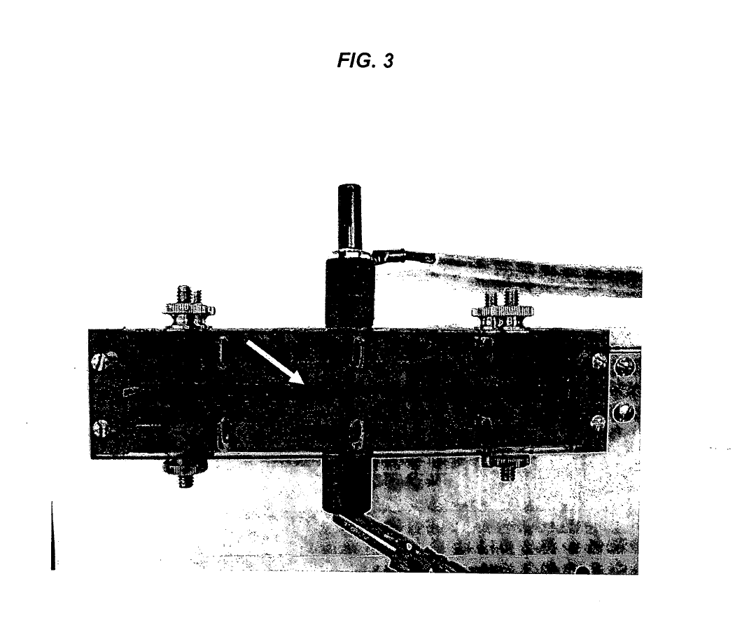

An APG plasma discharge was generated by a DB-APG in pure helium flowing gas. FIG. 1 depicts the experimental set up. Two parallel electrodes within a few millimeters of each other were supplied by a high-voltage (approximately 1 kV) power supply at high audio (˜10 kHz) frequency. Both electrodes were covered by a polycarbonate layer. FIG. 3 depicts the plasma region as a faint bluish glow (arrow).

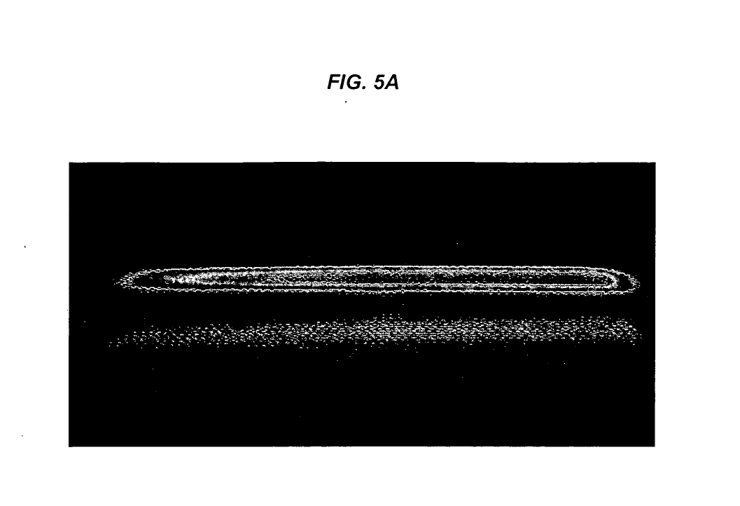

A time-averaged spectrum of an APG plasma discharge in pure helium was taken in the visible wavelength range of 663 nm to 786 nm, as seen in FIG. 4. A dominant helium electronic transition (3³S→2³P) at a wavelength of 706.5 mn was evident.

Plasma Stability And Uniformity

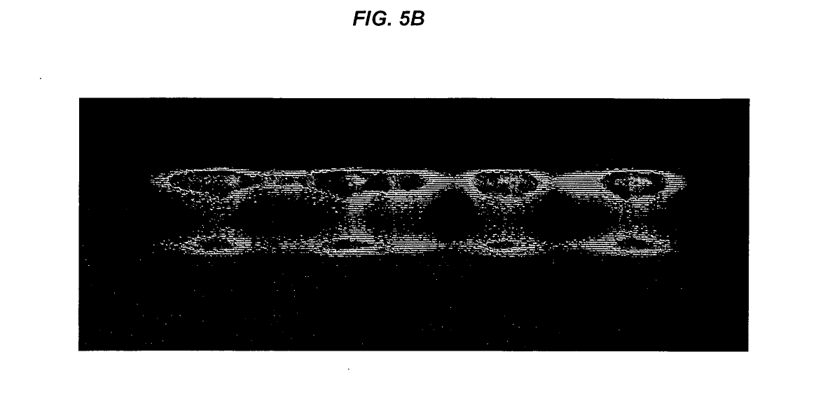

A uniform APG discharge was produced within APG stability boundaries. The discharge was imaged with an ICCD camera system with an exposure (gate) time of 300 ns. As shown in FIG. 5A, taken at an instant when the top electrode/dielectric was the momentary cathode, a relatively bright sheath was seen adjacent to the momentary cathode surface and a relatively weaker sheath was seen at the anode. FIG. 5A also shows that the discharge was clearly uniform across the entire electrode area, a property that is highly desirable for efficient, reproducible, and high-throughput particle processing.

The operating parameters of the APG discharge were then changed, so that the same discharge became unstable. As shown in FIG. 5B, groups of streamers and arc filaments destroyed the uniformity of the discharge, making it an undesirable plasma mode for surface processing.

### Example 2

### Surface Functionalization of Microparticles

Synthesis of Double Emulsion Particles

As a specific example embodiment, PLGA microparticles were synthesized using the double emulsion, solvent evaporation process described by Kasturi et al. (Mol. Ther., 2003, 7, S224). Three hundred fifty milligrams of PLGA microparticles, both without and with carboxylic acid (RG502 or RG502H, Boehringer Ingelheim, Virginia, Mw approximately 12,000 Da) end cap, were dissolved in 7 ml of methylene chloride (EMD Chemicals, New Jersey) to yield a 5% (weight/volume) polymer solution. Deionized water (300 μl) was added to the polymer solution to form a primary emulsion, which was then homogenized at 10,000 rpm for 2 min using a Silverson SL2T bench top homogenizer. The primary emulsion was then added to 50 ml of 1% PVA solution and homogenized for 1 min to obtain a w/o/w emulsion followed by solvent evaporation with magnetic stirring for 3 hours, to achieve microparticle formation and hardening. The microparticles were washed three times with deionized water, lyophilized for >16 hours, and stored at −20° C. for future use.

Scanning Electron Microscopy Analysis of Particles

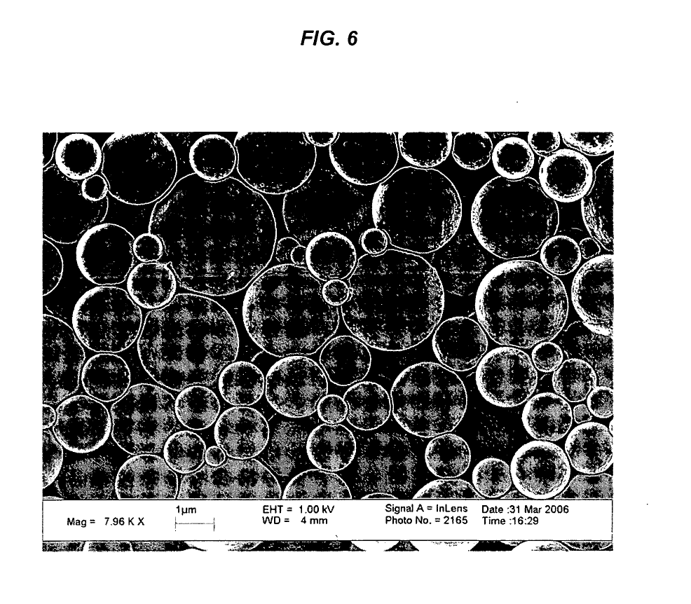

Scanning electron microscopy was used as an alternative technique to analyze the size distributions obtained using dynamic light scattering and also to verify any macroscopic changes in the morphology of these formulations. Microparticles were deposited on aluminum stubs obtained from the electron microscopy facility (Texas Material Institute (TMI), University of Texas at Austin, Austin, Tex.). Microparticle suspensions were made with 0.2 μm filtered purified water and allowed to air-dry overnight. The dried microparticle deposits on the aluminum stubs were sputter coated with 60:40 (gold:palladium) using the sputter coater at the core facility (TMI). Microparticles sputter coated with gold:palladium were visualized using a Leo 1530 Scanning Electron Microscope (Texas Material Institute (TMI), University of Texas at Austin, Austin, Tex.). As shown in FIG. 6, the microparticle formulation is a polydisperse particle mixture, with the particle have an average diameter of approximately 1.5 microns within a 5-micron size range.

Helium Glow Plasma Discharge-Based Treatment of PLGA Particles

The PLGA particles were suspended in deionized water at a concentration of 10 mg/ml, and 0.25 ml was evenly spread over the surface area of a frosted glass microscope slide. The area on the glass side was found to match the dimension of the electrode closely and thus the spreading of the suspension over the slide ensured plasma exposure of the entire surface of the slide over which the particles were deposited. The concentration was also varied to deposit 2.5 mg or 5 mg of particles on the slides. The suspensions deposited on the slides were air-dried overnight, stored at 4° C. before plasma exposure and modification.

The particle-laden slides were placed on one of the electrode-dielectric surfaces in an APG discharge chamber and immobilized with scotch tape. The slides were immobilized and centered to maximize plasma exposure. Pure helium APG plasma was generated in a 5-mm gap between the dielectric layers (FIG. 2). The electrode drive voltage was 1.7 kV with a 10 kHz frequency. The microparticles were exposed to the plasma for varying durations of time ranging from a few seconds to several minutes. The microparticles were subsequently removed from the surface by mechanically scraping them off the slide and suspended in 1 mM KCl. Their average charge state was measured using a zeta potential measurement instrument as described infra.

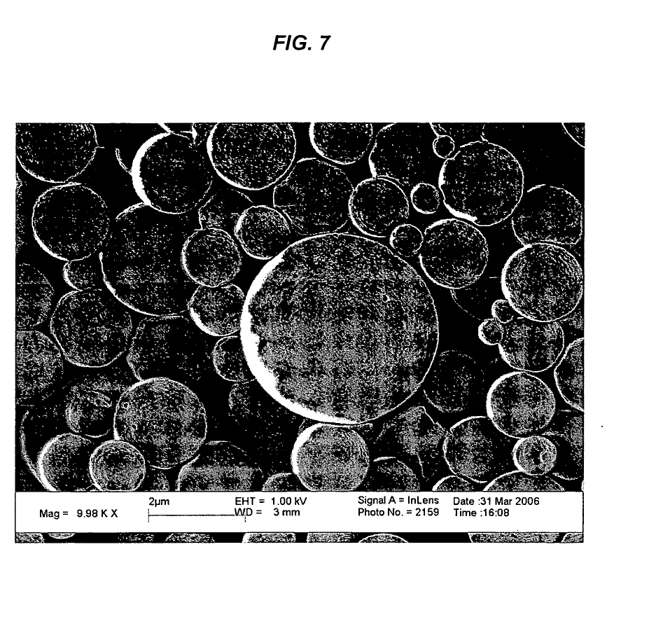

Using scanning electron microscopy as described supra, the plasma-modified PLGA particles were analyzed for size distribution and surface morphology. As shown in FIG. 7, the surface of the plasma-modified particles show no visible macroscopic change as compared to non-modified particles (cf. FIG. 6).

Zeta Potential Analysis of Plasma Discharge-Exposed Particle Formation

Zeta potential analysis was conducted using a Zeta Plus analyzer (BrookHaven Instruments Corp, Holtsville, N.Y.) to analyze the surface charge changes of the particles. Zeta potential analysis is a common technique employed to check for any surface charge changes for colloidal suspension. Lyophilized samples of unmodified and plasma-modified PLGA microparticle formulations were suspended in 1 mM KCl at 1 mg/ml concentration. Ten readings were noted per sample at the pre-set temperature of 25° C. The mean and standard error were noted for the distribution of charged particles. Results were replotted using Microsoft EXCEL eliminating any outliers (2× standard error in the 10 readings noted and the standard error were recalculated).

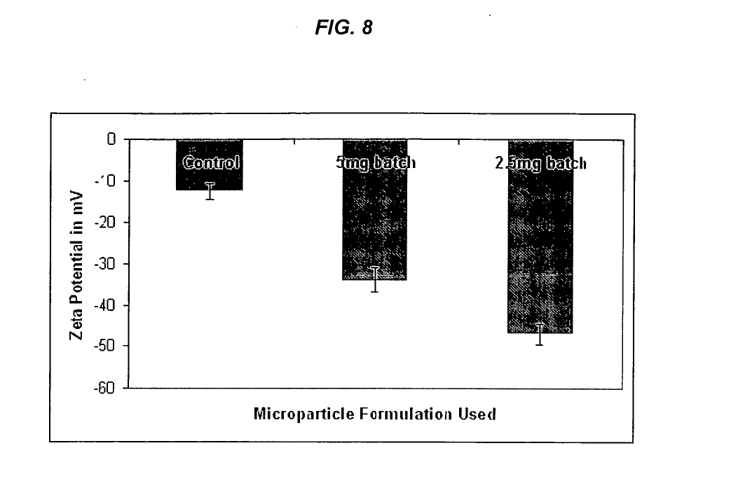

Zeta potential analysis showed that the helium plasma exposed PLGA microparticles were increasingly negative depending on the mass of microparticles deposited (FIG. 8). Five milligrams of microparticles deposited on the glass slides yielded small areas of clustered and other areas of multilayered particles. Decreasing the deposition mass to 2.5 mgs decreased the number of areas with multilayers and most areas appeared as spots of monolayer of microparticles (FIG. 8). The results of the zeta potential of unmodified and plasma modified microparticles are compiled as shown in Table 1.

Table 1 presents the zeta potential, protein loading and release of protein 24-hr post loading from plasma-modified PLGA microparticles. Protein release was analyzed 24-hr post incubation in physiologic conditions of PBS buffer at 37° C. All results are a reported mean of N=4 independent experiments done in triplicate each.

TABLE 1

| | Zeta Potential | Protein Loading | Protein Release |
|---|---|---|---|
| Formulation | (mV ± Std. Error) | (% loading) | (% release) |
| Unmodified | −13.03 ± 1.69 | 43.0 ± 13.24 | N.D. |
| PLGA | | | |
| 2.5 mg PLGA, | −42.02 ± 2.32 | 90.5 ± 10.25 | 20.67 ± 9.71 |
| plasma modified | | | |
| 5 mg PLGA, | −32.89 ± 5.45 | 79.25 ± 16.82 | 26.5 ± 0.071 |
| plasma modified | | | |

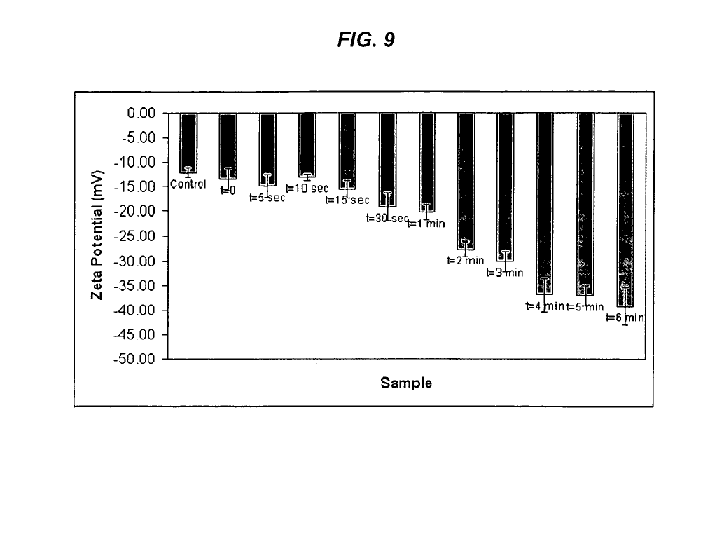

An affect of the exposure times of plasma gas discharge was studied as shown in FIG. 9. The zeta potentials of plasma-processed microparticles were increasingly negative with increased time of exposure of the particles to the plasma gas discharge. The control group of unprocessed particles had a small zeta potential of about −12 mV. The plasma-processed particles showed a consistent increase in the negative zeta potentials with increasing exposure time. The 1-minute exposure produced particles with about −20 mV zeta potentials, whereas the 6-minute exposure produced particles with about −40 mV zeta potentials.

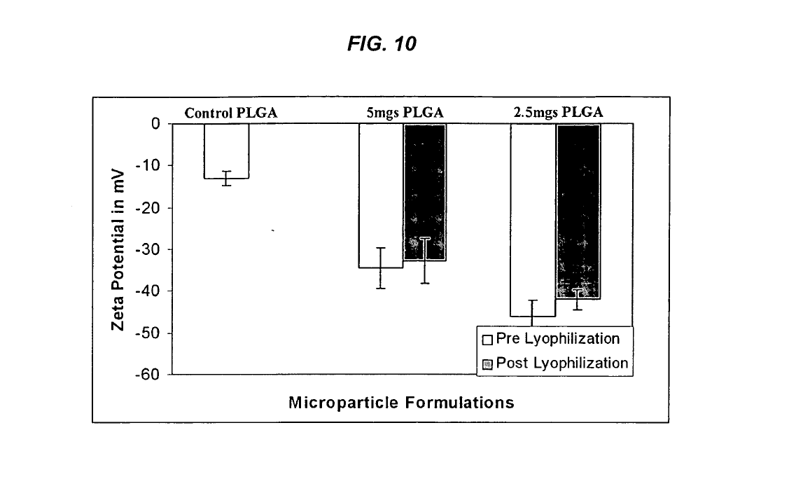

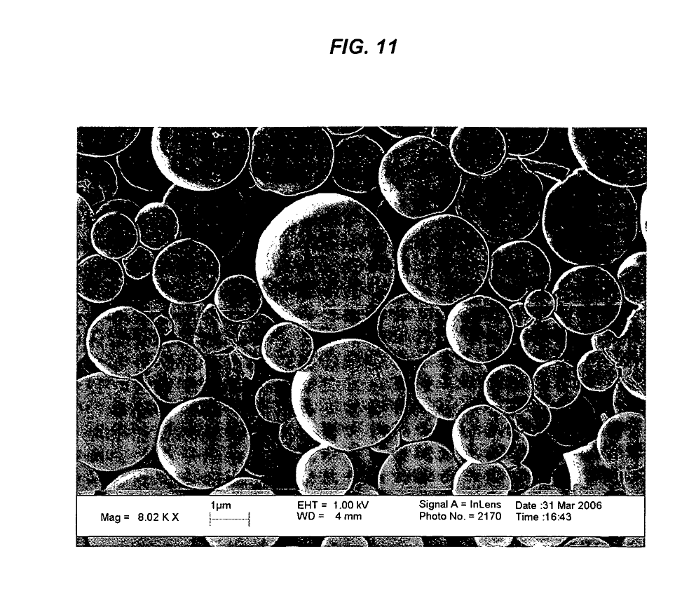

An important part of formulation synthesis is to create off the shelf, ready to use formulation or delivery systems. Thus, it is important to determine whether freeze drying would alter the plasma-discharge modified surface. As shown in FIG. 10, the zeta potential of the freeze dried plasma modified microparticles was not significantly reversed and these formulations retained anionicity sufficiently enough to adsorb proteins.

### Example 3

### Loading the Functionalized Particles With Bio-Active Substances

Protein Loading And Quantitation On Plasma-Modified PLGA Particles

Lyophilized plasma modified PLGA microparticles were used for adsorption of lysozyme protein. Lysozyme has been used as a model protein for adsorption experiments as reported by Singh et al., 2006, supra. Lysozyme protein was loaded at 1% (wt/wt) to the mass of the PLGA formulation used. Unmodified PLGA microparticles were used as controls for the loading experiment. Lysozyme (50 μg) from a stock lysozyme solution in pH 7.0 HEPES buffer (5 mg/ml) was added to 5 mg of unmodified and plasma modified PLGA microparticles suspension in pH 7.0 HEPES buffer under mild vortexing. The total volume for the protein adsorption process was 1 ml in a 1.5-ml microcentrifuge tube.

The protein/microparticle mixture was rotated on an end-to-end shaker (Barnstead International, Dubuque, Iowa) for 12 hr overnight at 4° C. Following protein adsorption, the microparticles were centrifuged at 4,000 rpm using a 5810R refrigerated Eppendorf centrifuge for 20 min. The supernatants were collected and analyzed for protein content using the BCA assay for protein estimation as per the instructions from Pierce Biotechnology (Rockford, Ill.). The standard curves were plotted at increasing concentrations with lysozyme protein. The supernatant samples from the protein loaded microparticles were used at a 1:1 dilution with the working reagent prepared from the BCA kit and the absorbance was read at 570 nm.

As shown in Table 1, plasma-modified microparticles adsorbed protein greater than twice as efficiently as the unmodified PLGA microparticles. Modifications of both 2.5 mg and 5 mg of microparticles, although yielding different absolute values of negativity, retained the increased advantage post plasma modification in adsorbing proteins with greater than twice the levels as compared to the unmodified batches. Experiments were repeated at least four independent times and the values of negativity post-plasma modification were found to be statistically significant compared to unmodified PLGA microparticles (P<0.05).

In Vitro Release of Protein From Anionic Microparticles

Protein loaded plasma modified microparticles were resuspended in 1× phosphate buffered saline (PBS) and rotated on an end-to-end shaker at 37° C. Microparticle suspensions were centrifuged at 3 hr and 24 hr at 8,000 rpm in a 5810R refrigerated centrifuge and the supernatants withdrawn and replaced with fresh buffer. The supernatants were analyzed for protein content using the BCA assay discussed above. The cumulative release of protein from the microparticles was calculated (Table 1).

As shown in Table 1, lysozyme is only released at early time points very similar to a burst release mechanism most typically seen with encapsulated designs for proteins with formulations. No increased or sustained release was noted beyond one day and samples were retrieved up to a week for analysis. It is presumed that extremely tight electrostatics could prevent further protein release in vitro but the situation in vivo could be more dynamic with competing anions facilitating release of proteins from the surface. Also, the degradation of the PLGA polymers as demonstrated by other research groups could ensure that proteins could come off eventually within as early as eight days (Thiele et al., 2003, Pharm. Res., 20, 221-8).

Scanning Electron Microscopy Analysis of Protein-Loaded Particles

Scanning electron microscopy was used to evaluate the size distributions obtained using dynamic light scattering and also to verify any macroscopic changes in the morphology of these formulations. As discussed supra, protein loaded microparticles were deposited on aluminum stubs obtained from the electron microscopy facility at the University of Texas at Austin. Microparticle suspensions were made with 0.2 μm filtered purified water and allowed to air-dry overnight. The dried microparticle deposits on the aluminum stubs were sputter coated with 60:40 (gold:palladium) using the sputter coater at the core facility (TMI). Microparticles sputter coated with gold:palladium were visualized using a Leo 1530 Scanning Electron Microscope.

In alternate example embodiment of the present invention, plasmid DNA encoding hepatitis B-surface antigen was used to test the efficacy with which the functionalized particles could be loaded with DNA drugs. The absorbance at 260 nm of the supernatant after particle separation was used as an indicator of whether DNA attached to the surface of the particles. Protein loading was similarly evaluated using a model antigen bovine serum (BSA).

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

All patents, patent applications, publications, products descriptions, and protocols, and references cited herein are incorporated by reference for all purposes, and specifically for a referenced method or procedure.

We claim:

1. A method of functionalizing a polymeric material, which comprises exposing a surface of a polymeric material to an atmospheric pressure glow plasma discharge under conditions wherein exposure to the plasma discharge functionalizes the surface of the polymeric material.

2. The method of claim 1, wherein the polymeric material is a biodegradable polymer.

3. The method of claim 2, wherein the biopolymer is poly (lactide-co-glycolide) (PGLA).

4. The method of claim 1, wherein the polymeric material is a particle or a scaffold.

5. The method of claim 1, which comprises:
entraining the polymeric material into a gas phase to form a suspended polymeric material; and
introducing the suspended polymeric material into a volume of the atmospheric pressure glow plasma discharge.

6. The method of claim 5, which further comprises collecting the suspended polymeric material from the atmospheric pressure glow plasma discharge.

7. The method of claim 6, which further comprises loading the surface of the functionalized polymeric material with a bioactive agent.

8. The method of claim 7, wherein the bioactive agent is a protein.

9. A composition comprising a polymeric material having a surface that has been exposed to an atmospheric pressure glow plasma discharge resulting in the presence of charges or charged radical groups on the surface of the polymeric material.

10. The composition of claim 9, which further comprises a bioactive agent associated with the charges or charged radical groups on the surface of the polymeric material.

11. The composition of claim 10, wherein the bioactive agent comprises a small molecule drug, a protein drug, a peptide drug, a DNA drug, a RNA drug, an oligonucleotide drug, an immunomodulatory agent, a vaccine antigen, or a contrast or imaging agent.

12. The composition of claim 10, wherein the bioactive agent is a protein.

13. The composition of claim 9, wherein the polymeric material is a biodegradable polymer.

14. A composition comprising a polymeric material having an interior and having a surface that has been exposed to an atmospheric pressure glow plasma discharge resulting in the presence of charges or charged radical groups on the surface of the polymeric material.

15. The composition of claim 14, which further comprises:
a first bioactive agent associated with the charges or charged radical groups on the surface of the polymeric material; and
a second bioactive substance disposed within the interior of the polymeric material.

16. The composition of claim 14, wherein the polymeric material is a biodegradable polymer.

17. The composition of claim 16, wherein the polymeric particle is a biodegradable particle of poly(lactide-co-glycolide).

18. The composition of claim 15, wherein the first bioactive agent and the second bioactive agent comprises a small molecule drug, a protein drug, a peptide drug, a DNA drug, a RNA drug, an oligonucleotide drug, an immunomodulatory agent, a vaccine antigen, or a contrast or imaging agent.

19. The composition of claim 15, wherein the first bioactive agent comprises a vaccine antigen; and the second bioactive agent comprises an immunomodulatory agent that regulates immune response to the vaccine antigen.

20. The composition of claim 15, wherein the polymeric material is a biopolymeric particle of poly(lactide-co-glycolide); the first bioactive agent comprises a vaccine, and the second bioactive agent comprises an immunomodulatory agent that regulates immune response to the vaccine.
